(19) United States
(12) Patent Application Publication
(10) Pub. No.: US 2017/0333050 A1
(43) Pub. Date: Nov. 23, 2017

(54) FASTENING FOR ELASTIC BAND

(71) Applicant: Silicamed Limited, Kowloon (HK)

(72) Inventor: Kenneth Thomas Mccabe, Kowloon (HK)

(21) Appl. No.: 15/225,186

(22) Filed: Aug. 1, 2016

(30) Foreign Application Priority Data
May 19, 2016 (GB) 1608856.9

(51) Int. Cl. A61B 17/132; A61B 17/00

(52) U.S. Cl. CPC A61B 17/1327; A61B 2017/00862

(57) ABSTRACT

A fastening for an elastic band comprising an attachment for a first end of the band and a cleat comprising a cleat slot into which a second end of the band can be jammed, and such a fastening in combination with an elastic band, which may constitute a tourniquet.

This invention relates to a fastening for an elastic band. It has particular utility as a fastening for an elastic tourniquet, but can have other applications.

Simple and inexpensive disposable tourniquets comprise an elastic band with a fastening at one end, the fastening being adapted to hold the other end when is has been wrapped and stretched around a limb, for example, an arm, in preparation for phlebotomy. Fastenings are known of more or less complexity, design for ease of fastening, often for single-handed fastening, and for fastening to a certain tension.

One such tourniquet comprises a band with perforations in an end one of which is located a button with a wide, flat base. In use, the band is wrapped around the limb and the button put through one of the perforations when the band is stretched to the desired tension. With perforations spaced apart, for example, every centimetre, the tension can be adjusted quite finely. Whilst the concept is simple and easy to manufacture, the need to drill closely spaced apertures along the band involves a certain manufacturing cost. Other tourniquets do not have perforations, but rely on a mechanical buckle or clamp that has at least one hinged section and a snap close action, which again, though simple in concept and design, nevertheless involves a certain manufacturing cost.

The present invention provides a fastening for an elastic band, such as a tourniquet, that is easy and inexpensive to manufacture, is easy both to apply and release, has no moving parts, and is easily adjustable for tension. The invention comprises a fastening for an elastic band comprising an attachment for a first end of the band and a cleat comprising a cleat slot into which a second end of the band can be jammed.

The fastening may comprise a flat base having a base slot comprising the attachment for the first end of the band and, upstanding from the base, two posts defining a slot adapted to receive and retain the second end of the band.

The two posts may define a slot extending at right angles to the flat base. The slot may widen where it adjoins the base, and may there have a triangular shape.

The base may be elliptical in shape, having a long axis and a short axis, with the base slot parallel to the long axis. The base slot may have a width substantially equal to half the width of the cleat slot.

The invention comprises such a fastening in combination with an elastic band attached in the base slot. The elastic band may have a thickness substantially equal to the width of the base slot, so that it is jammed in the base slot.

For a tourniquet, the long axis of an elliptical base may be 25 mm, the width of the slots may be 1 mm.

A fastening for an elastic band and a fastening in combination with an elastic band will now be described with reference to the accompanying drawings, in which:

FIG. 1 is a front elevation of the fastening;

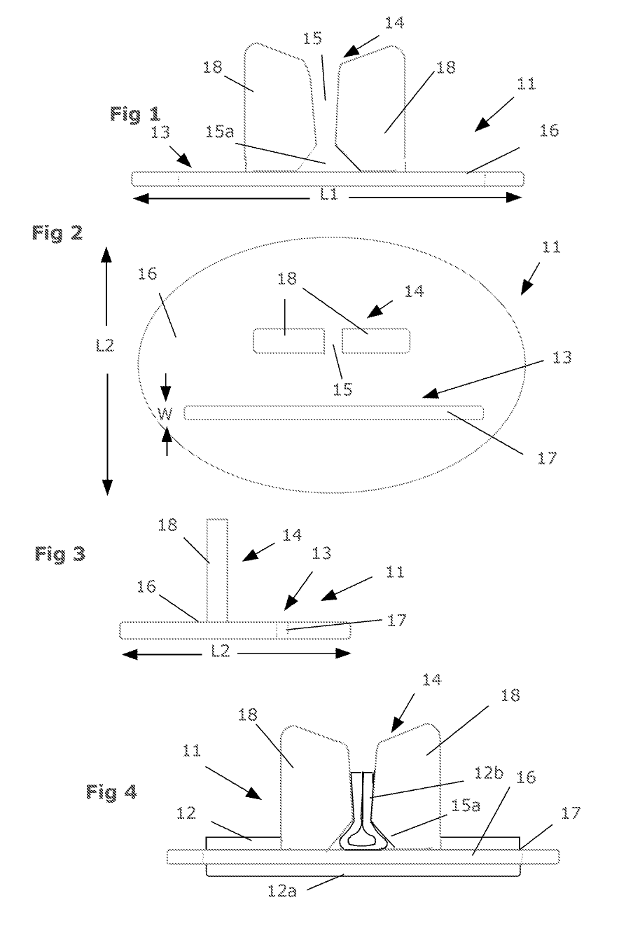

FIG. 2 is a plan view;

FIG. 3 is a side elevation;

FIG. 4 is a view like FIG. 1, showing a band in the fastening;

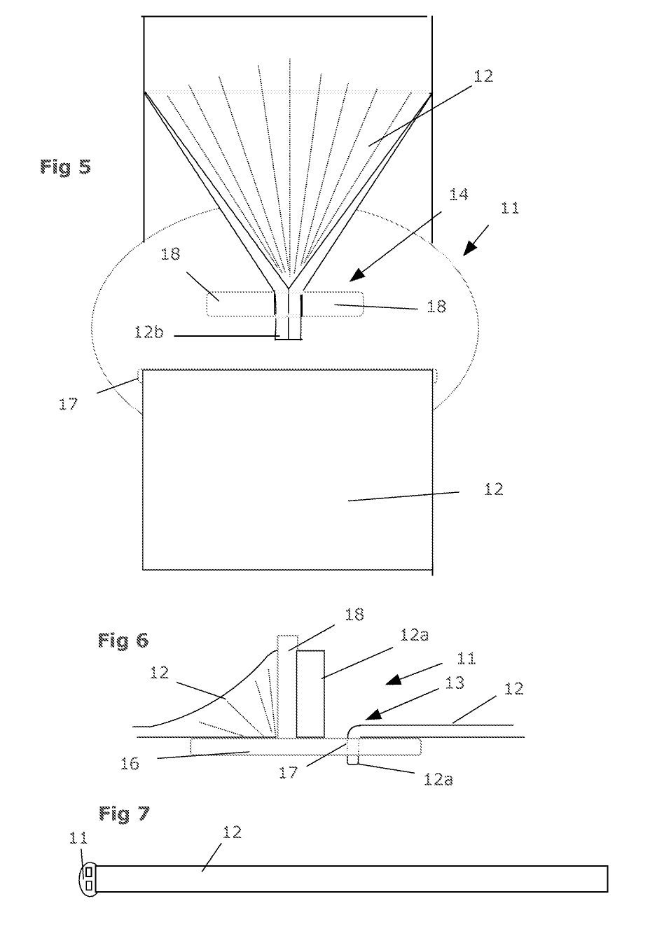

FIG. 5 is a view like FIG. 2, showing a band in the fastening;

FIG. 6 is a view like FIG. 3, showing a band in the fastening; and

FIG. 7 is a view of a tourniquet comprising a band attached in the fastening ready for use.

The drawings illustrate a fastening 11 for an elastic band 12 comprising an attachment 13 for a first end 12a of the band and a cleat 14 comprising a cleat slot 15 into which a second end 12b of the band can be jammed.

The fastening 11 comprises a flat base 16 having a base slot 17 comprising the attachment 13 for the first end 12a of the band 12 and, upstanding from the base 16, two posts 18 defining the cleat slot 15 adapted to receive and retain the second end 12b of the band.

The two posts 18 define the slot 15 extending at right angles to the flat base 16. The slot 15 widens where it adjoins the base 16, and there has a triangular shape.

The base 16 is elliptical in shape, having a long axis L1 and a short axis L2, with the base slot 17 parallel to the long axis L1. The base slot 17 has a width W substantially equal to half the width of the cleat slot 15. For a tourniquet such as is used on a patient's arm for phlebotomy, convenient dimensions are L1=25 mm, L2=19 mm and W=1 mm.

FIGS. 4, 5 and 6 illustrate the fastening 11 in combination with the end 12a of the elastic band 12 attached in the base slot 17. The elastic band 12 has a thickness substantially equal to the width W of the base slot 17, so that it is jammed in the base slot. The end 12a of the band 12 protrudes below the base slot 17, which arrangement balances torsion forces on the fastening when tightened.

The other end 12b of the band 12 is shown jammed in the cleat slot 15, folded double by being laid flat on top of the posts 18, the tops of which taper down towards the slot 15 so that the band folds into the slot 15 and jams therein. The triangular base profile 15a of which accommodates the fold in the band 12.

The band 12 will be provided of sufficient length to accommodate all limb sizes for which the tourniquet is intended. This will mean that in most cases, there will be a substantial length of band 12 emerging from the cleat slot 15, with, of course, sufficient to grip for stretching the band 12, but FIGS. 4, 5 and 6 show the band terminating just beyond the slot 15, for clarity's sake.

FIG. 7 shows a tourniquet comprising fastening 11 and attached band 12, ready for use.

The action of applying the tourniquet is greatly simplified by this construction—it is only required to hold the button against the patient with one hand, wrap the band 12 around the limb, and pull it down into the cleat slot 15.

Markings, not shown, on the band 12, as disclosed in WO12015/145330, which change shape as the band is stretched, help in judging when the tension is right.

1. A fastening for an elastic band comprising an attachment for a first end of the band and a cleat comprising a cleat slot into which a second end of the band can be jammed.

2. A fastening according to claim 1, comprising a flat base having a base slot comprising the attachment for the first end of the band and, upstanding from the base, two posts defining a slot adapted to receive and retain the second end of the band.

3. A fastening according to claim 2, in which the two posts define a slot extending at right angles to the flat base.

4. A fastening according to claim 2, in which the slot widens where it adjoins the base.

5. A fastening according to claim 4, in which the slot has a triangular shape where it widens.

6. A fastening according to claim 1, in which the base is elliptical in shape, having a long axis and a short axis, with the base slot parallel to the long axis.

7. A fastening according to claim 1, in which the base slot has a width substantially equal to half the width of the cleat slot.

8. A fastening according to claim 1, in combination with an elastic band attached in the base slot.

9. A combination according to claim 8, in which the elastic band has a thickness substantially equal to the width of the base slot, so that it is jammed in the base slot.

10. (canceled)
